SEQUENCE LISTING (13 sequences)

SEQ ID NO: 1 - protein, 1389 aa, Artificial Sequence
  nuclease-inactive Cas9
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGPPKKKRKVYPYDVPDYA

SEQ ID NO: 2 - DNA, 47 bp, Orientia Tsutsugamushi
tataaagatcttgttaaattgcagcgtcatgcaggaattaggaaagc

SEQ ID NO: 3 - DNA, 47 bp, Orientia Tsutsugamushi
gctttcctaattcctgcatgacgctgcaatttaacaagatctttata

SEQ ID NO: 4 - DNA, 47 bp, Bunyavirus
aaaaattagctgcccaacaagaagaagatgcaaagaatcaaggtgaa

SEQ ID NO: 5 - DNA, 47 bp, Bunyavirus
ttcaccttgattctttgcatcttcttcttgttgggcagctaattttt

SEQ ID NO: 6 - DNA, 20 bp, Artificial Sequence
  primer
cgagagagctggcctatgaa

SEQ ID NO: 7 - DNA, 20 bp, Artificial Sequence
  primer
ttccctgatgccttgacgat

SEQ ID NO: 8 - DNA, 20 bp, Artificial Sequence
  primer
gcagcagctgttaggctttt

SEQ ID NO: 9 - DNA, 20 bp, Artificial Sequence
  primer
ttgcagtcaccttcaccttg

SEQ ID NO: 10 - DNA, 32 bp, Artificial Sequence
  primer
ggaggcctactctctgtggcaagatgccttca

SEQ ID NO: 11 - DNA, 32 bp, Artificial Sequence
  primer
ggccttcagccactttacccgaacatcattgg

SEQ ID NO: 12 - DNA, 34 bp, Artificial Sequence
  primer
gcagcagcagctgttaggcttttaaatggcaatg

SEQ ID NO: 13 - DNA, 33 bp, Artificial Sequence
  primer
gctgcttgcagtcaccttcaccttgattctttg

(19) United States
(12) Patent Application Publication
(10) Pub. No.: US 2019/0203280 A1
(43) Pub. Date: Jul. 4, 2019

(54) COMPOSITION AND METHOD FOR IMPROVING SENSITIVITY AND SPECIFICITY OF DETECTION OF NUCLEIC ACIDS USING DCAS9 PROTEIN AND GRNA BINDING TO TARGET NUCLEIC ACID SEQUENCE

(71) Applicants: UNIVERSITY OF ULSAN FOUNDATION FOR INDUSTRY COOPERATION, Ulsan (KR); THE ASAN FOUNDATION, Seoul (KR)

(72) Inventors: Yong SHIN, Seoul (KR); Yong Sub KIM, Seoul (KR)

(73) Assignees: UNIVERSITY OF ULSAN FOUNDATION FOR INDUSTRY COOPERATION, Ulsan (KR); THE ASAN FOUNDATION, Seoul (KR)

(21) Appl. No.: 16/331,148

(22) PCT Filed: Sep. 6, 2017
(86) PCT No.: PCT/KR2017/009765

§ 371 Date: Mar. 7, 2019

(30) Foreign Application Priority Data
Sep. 7, 2016 (KR) 10-2016-0115024
Sep. 4, 2017 (KR) 10-2017-0112474

(51) Int. Cl. C12Q 1/6848; C12Q 1/06; C12Q 1/689; C12Q 1/70; C12Q 1/6853; C12Q 1/6811; C12Q 1/6806; C12Q 1/686; C07H 21/02

(52) U.S. Cl. CPC C12Q 1/6848; C12Q 1/06; C12Q 1/689; C12Q 1/70; C12Y 207/07006; C12Q 1/6811; C12Q 1/6806; C12Q 1/686; C07H 21/02; C12Q 1/6853

(57) ABSTRACT

A composition and a method for improving sensitivity and specificity of detection of nucleic acids, use dCas9 and gRNA, which binds to a target nucleic acid sequence, in amplification of nucleic acids including DNAs, RNAs, and the like increases efficiency in amplification of nucleic acids and thereby can ultimately improve sensitivity and specificity of a target diagnosis on the basis of a functional difference between Cas9 and dCas9.

### TECHNICAL FIELD

The present invention relates to a composition and a method of improving the sensitivity and specificity of nucleic acid detection using dCas9 protein and gRNA binding to a target nucleic acid sequence.

### BACKGROUND ART

Methods for labeling and detecting nucleic acids which are difficult to detect in their natural state have been applied to various fields of molecular biology and cell biology. In order to detect signals in Southern blotting, Northern blotting, in situ hybridization and nucleic acid microarray using a specific hybridization reaction, nucleic acid to which labeling substance is attached has been widely used. A method of amplifying DNA and labeling DNA at the same time using a labeled monomer (labeled dNTP) or a labeled primer in a polymerase chain reaction (PCR) is known. The DNA thus labeled can be detected by a microarray.

The method of labeling the nucleic acid at the same time as the PCR has an advantage in that a separate step for labeling is not required, but when the monomer labeled with a fluorescent dye or the like is used, it has lower PCR efficiency than the case of using the unlabeled monomer. In addition, since RNA cannot be amplified by the PCR method, a step of preparing cDNA by reverse transcription is required so as to detect the RNA by the FOR-labeled method, and especially, when the length is short like microRNA (miRNA), it has a problem that cDNA production is cumbersome. Accordingly, there is an urgent need to develop a nucleic acid detection technique having improved sensitivity and specificity.

The above-described methods are easy to detect a target nucleic acid when a large amount of the detected nucleic acid is retained. Although it is still widely used today, when a small amount of target nucleic acid is present, it is very difficult to detect it (low sensitivity), and frequently, it cannot detect only a specific target and erroneously it detect a non-specific target, due to other inhibitors (low specificity).

Sensitivity and specificity are very low due to more inhibitors when directly detecting in body fluids of patients such as blood and urine, etc. Newly launched viruses or bacteria nowadays worldwide require early diagnosis and treatment, so methods that can detect it quickly and accurately are needed. Because in new variant diseases, target factors are present in very small amounts, development of a more sensitive and specific method is urgent.

In addition to this, it is necessary to apply the process having improved sensitivity and specificity to method of identifying mutations of biomarkers that cause cancer. It is important to develop technologies that can increase sensitivity and specificity by combining conventional technologies as much as the development of new technologies with high sensitivity and specificity.

### DISCLOSURE

### Technical Problem

An object of the present invention is to provide a composition for improving the sensitivity and specificity of nucleic acid detection, comprising dCas9 (dead Cas, nuclease-inactive Cas9) protein and gRNA (guide RNA) binding to a target nucleic acid sequence, as an active ingredient and a method of improving the sensitivity and specificity of nucleic acid detection using the same.

### Technical Solution

In order to accomplish the above object, the present invention provides a composition for improving sensitivity and specificity of nucleic acid detection, comprising: protein binding to a target nucleic acid sequence; or a complex of the protein and a gRNA (guide RNA) binding thereto, as an active ingredient.

Also, the present invention provides a composition for improving sensitivity and specificity of nucleic acid detection, comprising dCas9 (dead Cas, nuclease-inactive Cas9) protein and gRNA (guide RNA) binding to a target nucleic acid sequence, as an active ingredient.

In addition the present invention provides a kit for improving sensitivity and specificity of nucleic acid detection, comprising dCas9 (dead Cas, nuclease-inactive Cas9) protein and gRNA (guide RNA) binding to a target nucleic acid sequence, as an active ingredient.

Furthermore, the present invention provides a method of improving the sensitivity and specificity of nucleic acid detection, comprising amplifying a target nucleic acid by adding a dCas9 protein and a gRNA (guide RNA) binding to a target nucleic acid sequence to a sample containing the target nucleic acid; and detecting an amplified target nucleic acid amplification product.

### Advantageous Effect

The present invention relates to a composition and a method for improving the sensitivity and specificity of nucleic acid detection using a dCas9 protein and a gRNA binding to a target nucleic acid sequence. Based on the functional difference between Cas9 having binding and cleavage function and dCas9 having only binding function and inactive cleavage function, when dCas9 protein and gRNA binding to a target nucleic acid sequence are used for nucleic acid amplification such as DNA and RNA, the efficiency of nucleic acid amplification is increased thereby ultimately achieving excellent sensitivity and specificity of the target diagnosis.

In addition, when the composition for improving the sensitivity and specificity of nucleic acid detection using dCas9 protein and gRNA binding to a target nucleic acid sequence according to the present invention is applied to a biosensor, the amplification efficiency is almost twice as high as that of the prior one and the difference between non-target and target increases by at least four times. In particular, since non-specific amplification of non-target is prevented, sensitivity and specificity are improved remarkably and therefore, the present invention can be utilized for nucleic acid detection with improved sensitivity and specificity.

### DESCRIPTION OF DRAWINGS

FIG. 1 shows a schematic diagram of a CRISPR-mediated biosensor according to the present invention (SMR sensor: silicon microring resonator sensor, with dCas9: using dCas9, No dCas9: dCas9 unused).

FIG. 2 and FIG. 3 illustrate schematic diagrams of gRNAs design prepared for detection of Orientia tsutsugamushi (OT), a causative organism of scrub typhus (ST) and Bunyavirus, a causative organism of severe fever with thrombocytopenia syndrome (SFTS) (DsDNA: double stranded DNA, ssRNA: single stranded RNA, target region highlighted in blue and PAM sequence shown in red).

FIG. 4 illustrates in vitro cleavage analysis for confirming gRNA activity under buffer conditions indicating that only when the gRNA is matched to the target PCR product, Cas9 RNP cleaves the PRC product in both RPA buffer and NEBuffer 3.1 conditions (ST: scrub typhus, SFTS: severe fever with thrombocytopenia syndrome).

FIG. 5 shows the results of EMSA analysis using dCas9 RNP and 5′-biotinylated DNA duplexes, indicating that the target DNA duplexes were not cleaved but transferred by matched gRNAs in both RPA buffer and NEBuffer 3.1 conditions (ST: scrub typhus, Ctrl: control without target nucleic acid, sgRNA: single guide RNA).

FIG. 6 shows the result of ST-DNA detection within 30 min of the CRISPR-mediated biosensor (ST: SMR biosensor alone, ST with dCas9 RNP: dCas9-treated SMR biosensor, ST with Cas9 RNP: Cas9-treated SMR biosensor) (ST: scrub typhus, ST with dCas9 RNP: ST detection using dCas9, ST with Cas9 RNP: No dCas9: dCas9-unused ST detection).

FIG. 7 shows the resonance wavelength shift result for ST detection within 15 minutes of a CRISPR-mediated biosensor (SMR biosensor alone: black, with dCas9 RNP: light gray, with Cas9 RNP: dark gray) (ST: scrub typhus, Empty: control group without target nucleic acid).

FIG. 8 shows the relative resonance wavelength shift result of a CRISPR-mediated biosensor for 30 minutes [ST: scrub typhus, dCas9 RNP; ST without dCas9 RNP, ST with 1×dCas9 RNP (100 ng dCas9+75 ng gRNA), ST with 3×dCas9 RNP (300 ng dCas9+225 ng gRNA), and ST with 5×dCas9 RNP (500 ng dCas9+375 ng gRNA)].

FIG. 9a shows the detection limit of dsDNA of ST according to the CRISPR-mediated biosensor (0.54 aM or less, grey), which is more sensitive than the SMR biosensor alone (black) (ST: scrub typhus, ST with dCas9 RNP: ST detection using dCas9 RNP).

FIG. 9b shows the detection limit of RNA of SFTS according to the CRISPR-mediated biosensor (0.63 aM or less, grey), which is more sensitive than the SMR biosensor alone (black) (SFTS: fever with thrombocytopenia syndrome, SFTS with dCas9 RNP: SFTS detection using dCas9 RNP),

FIG. 10 shows the detection limit of real-time PCR for DNA and the detection limit of real-time RT-PCR for RNA, indicating that (a) real-time PCR showed a linear correlation between the concentration of the target DNA and the Ct value of the fluorescence signal, and a low concentration (<100 copies/ml) of target DNA was not detected (over 40 Ct value) and (b) real-time RT-PCR showed a linear correlation between the concentration of the target RNA and the Ct value of the fluorescence signal, and a low concentration (<100 copies/ml) of target RNA was not detected (over 40 Ct value) (ST standard curve, SFTS standard curve).

FIG. 11a shows high sensitive and specific detection of ST (gray) from a clinical sample according to a CRISPR-mediated biosensor, which was detected more sensitively and specifically than the SMR biosensor alone (black) (P1-3: positive as serum of ST patient, N1-3: negative as serum of SFTS patient).

FIG. 11b high sensitive and specific detection of SFTS-RNA (gray) from a clinical sample according to a CRISPR-mediated biosensor, which was detected more sensitively and specifically than the SMR biosensor alone (black) (P1-3: positive as serum of SFTS patient, N1-3: negative as serum of ST patient).

### BEST MODE

The present invention provides a composition for improving sensitivity and specificity of nucleic acid detection, comprising protein binding to a target nucleic acid sequence; or a complex of the protein and a gRNA (guide RNA) binding thereto, as an active ingredient.

In particular, the protein binding to the target nucleic acid sequence may be a zinc finger protein or a transcription activator-like effector protein, but it is not limited thereto.

Specifically, a complex of the protein and a gRNA (guide RNA) binding thereto may be a complex of dCas9 (dead Cas, nuclease-inactive Cas9) protein and a gRNA binding thereto or a complex of dCpf1 (dead Cpf1, nuclease-inactive Cpf1) protein and a gRNA binding thereto, but it is not limited thereto.

Most preferably, the present invention can comprise a complex of dCas9 (dead cas, nuclease-inactive Cas9) protein and a gRNA binding thereto as an active ingredient and the sensitivity and the specificity of nucleic acid detection are greatly improved.

Accordingly, the present invention provides a composition for improving sensitivity and specificity of nucleic acid detection, comprising dCas9 (dead Cas, nuclease-inactive Cas9) protein and gRNA (guide RNA) binding to a target nucleic acid sequence, as an active ingredient. Preferably, the dCas9 protein may be represented by the amino acid sequence of SEQ ID NO: 1, but it is not limited thereto.

The target may be any one of causative organism of an infectious disease selected from the group consisting of Orientia tsutsugamushi (OT), Bunyavirus, Mycobacterium tuberculosis, Mers virus and respiratory virus, but it is limited thereto.

In particular, the composition may further comprise a nucleic acid polymerase, a primer capable of amplifying the target nucleic acid and a buffer solution.

In detail, the nucleic acid is not particularly limited, but may be any DNA or RNA, and may be chromosomal DNA, mitochondrial DNA, mRNA, rRNA, tRNA, miRNA, cfDNA, cfRNA, ctDNA and the like, which are present in the cell.

The ‘dCas9’ of the present invention is a variant of Cas9 in which the 10th aspartic acid is changed to alanine and the 840th histidine is changed to alanine among amino acids thereof, thereby suppressing nuclease activity. According to previous report, Cas9 WT and dCas9 were treated with gRNA and DNA together and then subjected to electrophoresis, resulting that the Cas9 WT cuts the target DNA thereby obtaining the cleavage fragment but dCas9 does not obtain the cleavage fragment. This shows that nuclease activity of dCas9 is inhibited (Conformational control of DNA target cleavage by CRISPR-Cas9, Nature 527,110-113).

Also, the present invention provides a kit for improving sensitivity and specificity of nucleic acid detection comprising dCas9 (dead cas, nuclease-inactive Cas9) protein and gRNA (guide RNA) binding to a target nucleic acid sequence as an active ingredient.

In addition, the present invention provides a method of improving sensitivity and specificity of nucleic acid detection, comprising amplifying a target nucleic acid by adding a dCas9 protein and a gRNA (guide RNA) binding to a target nucleic acid sequence to a sample containing the target nucleic acid; and detecting an amplified target nucleic acid amplification product. Preferably, the dCas9 protein may be represented by an amino acid sequence of SEQ ID NO: 1, but it is not limited thereto.

In detail, the step of amplifying the target nucleic acid is not particularly limited as long as it can amplify the target nucleic acid, however, the target nucleic acid may be amplified by using PCR, real-time PCR (RT-PCR), reverse transcriptase PCR, isothermal nucleic acid amplification and Silicon Microring Resonator (SMR), etc.

Specifically, the nucleic acid is not particularly limited, but may be any DNA or RNA, and may be chromosomal DNA, mitochondrial DNA, mRNA, rRNA, tRNA, miRNA and the like, which are present in the cell.

Meanwhile, the amplified product can be detected by methods known in the art, for example, gel electrophoresis, ELGA (enzyme-linked gel assay), ECL (electrochromiluminescent), fluorescent material, radioactive isotopes and the like can be used.

Examples of the fluorescent material include a rhodamine including rhodamine, TAMRA, etc.; fluorescein including fluorescein, FITC (fluorescein isothiocyanate) and FAM (fluorecein amidite), etc.; bodipy (boron-dipyrromethene); alexa fluor; and cyanine including Cy3, Cy5, Cy7, indocyanine green, but it is not limited thereto.

Examples of the radioisotope include H-3, C-14, P-32, S-35, C1-36, Cr-51, Co-57, Co-58, Cu-64, Fe-59, Y-90, 1-124, 1-125, Re-186, 1-131, Tc-99m, Mo-99, P-32, CR-51, Ca-45, Ca-68, etc. but particularly it is not limited thereto.

Hereinafter, the present invention will be described in detail with reference to the following examples. It should be noted, however, that the following examples are illustrative of the present invention and are not intended to limit the scope of the present invention. The examples of the present invention are provided to more fully describe the present invention to those skilled in the art.

### EXPERIMENTAL EXAMPLE

The following experimental examples are intended to provide experimental examples that are commonly applied to the respective examples according to the present invention.

1. Protein Purification

For recombinant dCas9 RNP (ribonucleoprotein) purification, T7 Express BL21 (DE3) E. coli cells were transformed with the pET28a-His6-dCas9 plasmid. E. coli was cultured in Luria-Bertani (LB) broth at 30° C. until the OD600 reached 0.5-0.7 and then cultured in 0.2 mM isopropyl β-d-l-thiogalactopyranoside (IPTG) to induce protein expression. Cell pellets were obtained by centrifuging at 5,000 g and eluting by sonication in elution buffer [50 mM NaH₂PO₄, 300 mM NaCl, 10 mM imidazole (pH 8.0), 1 mM PMSF, 1 mM DTT, 1 mg/mL lysozyme]. The aqueous eluent was obtained by centrifugation at 8,000 g and reacted with Ni-NTA agarose beads for 1-2 hours (Qiagen). Protein-bound Ni-NTA agarose beads were washed with a washing solution [50 mM NaH₂PO₄, 300 mM NaCl, 20 mM imidazole (pH 8.0)] and the dCas9 protein was eluted with imidazole containing buffer [50 mM NaH₂PO₄, 300 mM NaCl, 250 mM Imidazole (pH 8.0)]. Protein buffer eluted with 100 K Amicon centrifugal filter (Millipore) was ion-exchanged, concentrated and analyzed with 4-12% Bis-Tris gel (ThermoFisher).

2. In Vitro Cleavage Analysis

The PCR product (400 ng) containing each of the Orientia Tsutsugamushi (OT) and severe fever with thrombocytopenia syndrome (SFTS) DNA sequences was mixed with 5.9 μl of rehydration buffer and 0.5 μl of 280 mM magnesium acetate (MgAc)) solution [provided by TwistAmp Basic RT kit]. 10 μl of the PCR product mixed with the buffer was reacted with 1 μg of Cas9 protein and 750 ng of sgRNA at 37° C. for 1 hour. The same PCR products as the positive control of cleavage assays were cleaved with 1× Buffer 3.1 (New England BioLabs) conditions. RNase A (4 μg) was added to remove sgRNA and the final product was analyzed by agarose gel electrophoresis.

3. In Vitro Binding Assay

The dsDNA template was prepared by annealing the 5′-biotinylated target DNA strand and the non-biotinylated non-target DNA strand at 1:1.5 molar ratio. Here, the respective sequences are as follows:

1) 5′-biotinylated target DNA strand of Orientia
Tsutsugamushi
OT_1_F_biotin:
(SEQ ID NO: 2)
TATAAAGATCTTGTTAAATTGCAGCGTCATGCAGGAATTAGGAAAGC
2) non-biotinylated non-target DNA strand of
Orientia Tsutsugamushi
OT_1_R:
(SEQ ID NO: 3)
GCTTTCCTAATTCCTGCATGACGCTGCAATTTAACAAGATCTTTATA
3) 5′-biotinylated target DNA strand of SFTS
SFTS_F_biotin:
(SEQ ID NO: 4)
AAAAATTAGCTGCCCAACAAGAAGAAGATGCAAAGAATCAAGGTGAA
4) non-biotinylated non-target DNA strand of SFTS
SFTS_R:
(SEQ ID NO: 5)
TTCACCTTGATTCTTTGCATCTTCTTCTTGTTGGGCAGCTAATTTTT

10 nM dsDNA was reacted with 300 nM dCas9 and 1 μM sgRNA under cleavage buffer conditions. After reacting at 37° C. for 20 minutes, the cells were treated with 10% TBE gel using 0.5×TBE buffer supplemented with 5 mM MgCl₂. The in vitro binding state was analyzed by electrophoretic mobility analysis (EMSA) according to the manufacturers instructions using chemiluminescent nucleic acid detection module kit (ThermoFisher) and Biodyne B nylon membrane (ThermoFisher).

4. Construction of Silicon Microring Resonator (SMR)

SMR and RPA (Recombinase Polymerase Amplification) were constructed and operated according to a conventionally known method so as to use SMR biosensor as a detection system for detecting target nucleic acid.

First, an SMR biosensor was treated with oxygen plasma cleaning (electric power: 100W, O₂: 80 sccm) for 1 minute and was soaked in 2% 3-aminopropyltriethoxysilane (APTES) dissolved in 95% ethanol at room temperature for 2 hours. The SMR biosensor was then cured at 120° C. for 15 minutes. Thereafter, the SMR biosensor was reacted with 2.5% glutaraldehyde (GAD) dissolved in deionized water containing 10 mM sodium cyanoborohydride for 1 hour at room temperature, rinsed with deionized water and dried under high purity nitrogen gas. Next, in order to immobilize the target primer on the SMR biosensor, the biosensor was reacted with the target primer dissolved in PBS (1 mM) containing 20 mM sodium cyanoborohydride solution for 16 hours at room temperature and rinsed with PBS to remove unbound target primers. At this time, an amine group was introduced at 5′-position of the target primer. Primers for detection of ST and SFTS were designed using the SFTS-S fragment and the ST-56-kDa type-specific gene, respectively (Table 1).

TABLE 1

| Analysis method | name | Sequence (5′-3′) | SEQ ID NO |
|---|---|---|---|
| RT-PCR | SFTS-F | CGAGAGAGCTGGCCTATGAA | SEQ ID NO: 6 |
| | SFTS-R | TTCCCTGATGCCTTGACGAT | SEQ ID NO: 7 |
| | ST-F | GCAGCAGCTGTTAGGCTTTT | SEQ ID NO: 8 |
| | ST-R | TTGCAGTCACCTTCACCTTG | SEQ ID NO: 9 |
| SMR biosensor | SFTS-F | GGAGGCCTACTCTCTGTGGCAAGATGCCTTCA | SEQ ID NO: 10 |
| | SFTS-R | GGCCTTCAGCCACTTTACCCGAACATCATTGG | SEQ ID NO: 11 |
| | ST-F | GCAGCAGCAGCTGTTAGGCTTTTAAATGGCAATG | SEQ ID NO: 12 |
| | ST-R | GCTGCTTGCAGTCACCTTCACCTTGATTCTTTG | SEQ ID NO: 13 |

5. SMR Biosensor Alone and CRISPR-Mediated Biosensor Operation

RPA and RT-RPA solutions were prepared respectively to amplify and detect the target nucleic acid using SMR biosensor alone. To prepare the RPA and RT-RPA solutions, 29.5 μl of rehydration buffer, 15 μl of RNase inhibitor and water and 2.5 μl of each 10 μM primer were mixed. The reaction mixture was then added to the lyophilized enzyme and 2.5 μl of a 280 mM magnesium acetate (MgAc) solution was dispensed into the cap of each tube, was added. Unidirectional shaking blending was performed for homogeneous dispensing. After mixing, 50 μl of the reaction buffer was divided into 10 μl droplets five times. To initiate the reaction for detection, 5 μl of the nucleic acid extracted from the patient's serum and 3 μl of dCas9 RNP (300 ng dCas9 and 225 ng gRNA) were added to each 10 μl reaction liquid droplets, and the biosensor was placed on a thermo electric cooler (TEC, Alpha Omega Instruments) equipped with a controller and a constant DC voltage was applied and maintained at a constant temperature (38° C. for DNA and 43° C. for RNA). The resonance spectrum of the biosensor was measured immediately, and the reference was used to obtain the baseline. The wavelength shift was monitored every 5 minutes up to 30 minutes and the amplification of the target nucleic acid was monitored in an unlabeled and real time manner. The relative resonance wavelength shift was calculated by the following equation.

ΔΔpm=(target wavelength value, pm)−(non-target wavelength value, pm)

6. Extraction and Preparation of Nucleic Acid Samples

Viral RNA was extracted from SFTS samples using QIAamp Viral RNA Kit (Qiagen Inc., Chatsworth, Calif., USA) and genomic DNA was extracted from ST samples using QIAamp DNA mini kit (Qiagen). To prepare a SFTS viral RNA transcriptome control, an RNA fragment containing the target region was amplified with a primer containing the T7 promoter sequence on the antisense strand. Amplification products were transcribed in vitro using the MEGAscript T7 Transcription Kit (Ambion Life Technologies, Carlsbad, Calif., USA). The synthetic RNA transcriptomes were purified using MEGAclear Kit (Ambion) and quantified with a Nanodrop spectrophotometer (Thermo Scientific, Waltham, Mass., USA). To prepare the ST bacterial DNA control, a DNA fragment containing the target region was amplified by PCR. The amplified DNA fragments were quantified with a Nanodrop spectrophotometer (Thermo Scientific, Waltham, Mass., USA).

7. Real-time PCR and Real-Time RT-PCR Analysis

The target DNA used as a template was obtained from clinical samples and real-time PCR and real-time RT-PCR analysis were performed using the primers shown in Table 1. Real-time PCR was performed with a denaturation step at 95° C. for 15 minutes, 45 cycles of 30 seconds at 95° C., 30 seconds at 55° C., and 30 seconds at 72° C., and a final extension step at 72° C. for 10 minutes. The target DNA (5 μl) was amplified in a total 20 μl of the reactant [2× brilliant SYBR green RT-qPCR master mix and 25 pmol of each primer].

And real-time RT-PCR analysis was performed by modifying the AriaMx (Aligent) Instrument protocol as follows. Namely, the target RNA (5 μl) was amplified in a total 20 μl of the reactant [2× brilliant SYBR green RT-qPCR master mix and 25 pmol of each primer]. The initial cDNA synthesis step was carried out for 20 minutes at 50° C., 15 cycles of 15 minutes at 95° C., 15 seconds at 95° C., 20 seconds at 55° C., and 20 seconds at 72° C. and a cooling step at 40° C. for 30 seconds. SYBR Green signal of amplification product was obtained using AriaMx Real-Time PCR System (Agilent).

8. Clinical Sample Preparation

ST and SFTS serum samples were collected from patients at the Asan Medical Center. For SFTS, viral RNA was detected by real-time RT-PCR in serum using DiaStar 2× OneStep RT-PCR Pre-Mix kit (SolGent, Daejeon, South Korea). ST diagnosis was determined by confirming the single positive result of Immunofluorescence analysis (IFA; SD Bioline Tsutsugamushi Assay; Standard Diagnostics, Yongin, South Korea) or ≥1: 640 or 4-fold increase of IFA titre in continuous samples. These protocols were approved by IRB (Institutional Review Board) of the Asan Medical Center and proceeded with consent from all participants.

### Example 1

### Sensitivity and Specificity Analysis of CRISPR-Mediated Biosensor

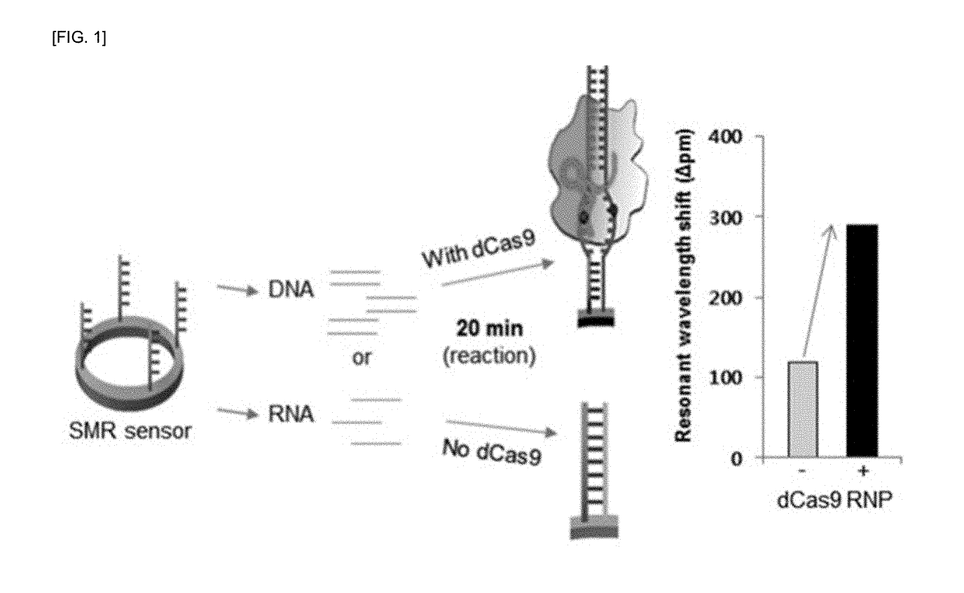

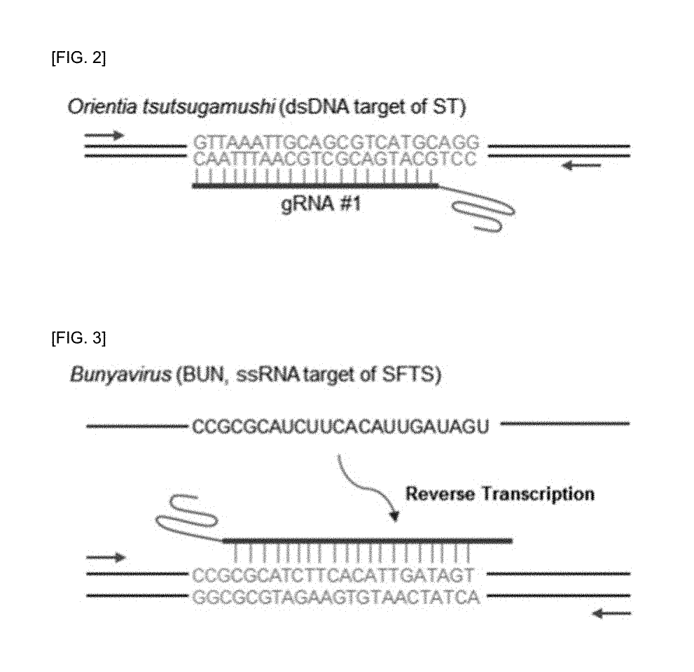

As shown in FIG. 1, pathogenic nucleic acid could be detected by using the combination of CRISPR/dCas9 and SMR biosensor from clinical samples in real time without any labeling. In order to detect these pathogenic nucleic acids, gRNAs targeting Orientia tsutsugamushi (OT), a causative organism of the scrub typhus (ST), and tuberculosis virus, which causes the severe fever with thrombocytopenia syndrome (SFTS) were prepared, respectively (FIG. 2 and FIG. 3).

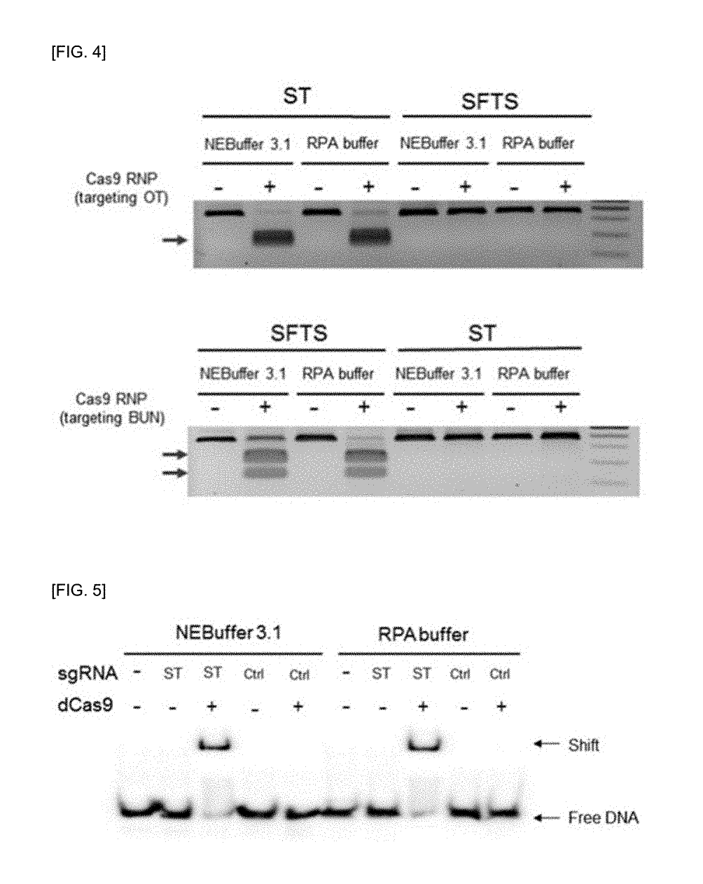

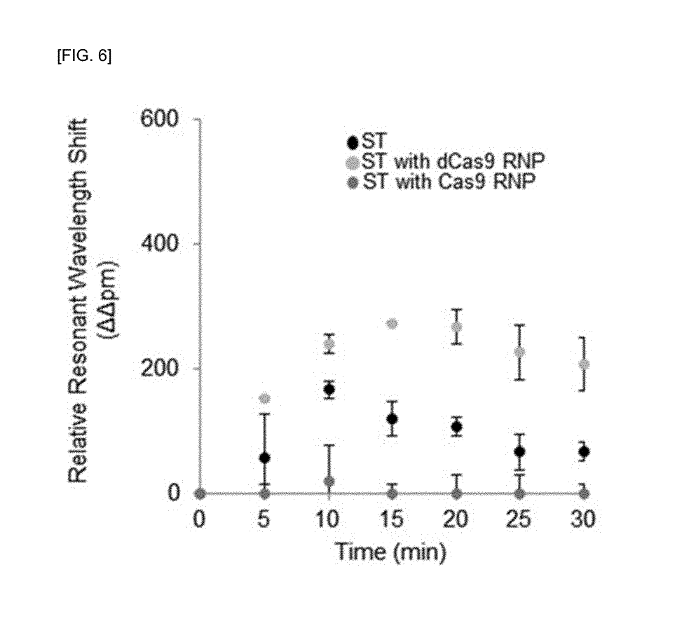

Through in vitro cleavage analysis and electrophoretic mobility analysis (EMSA), in RPA buffer, it was observed that the cleavage of the target DNA was induced in Cas9 RNP and the target DNA was bound to dCas9 RNP (FIG. 4 and FIG. 5).

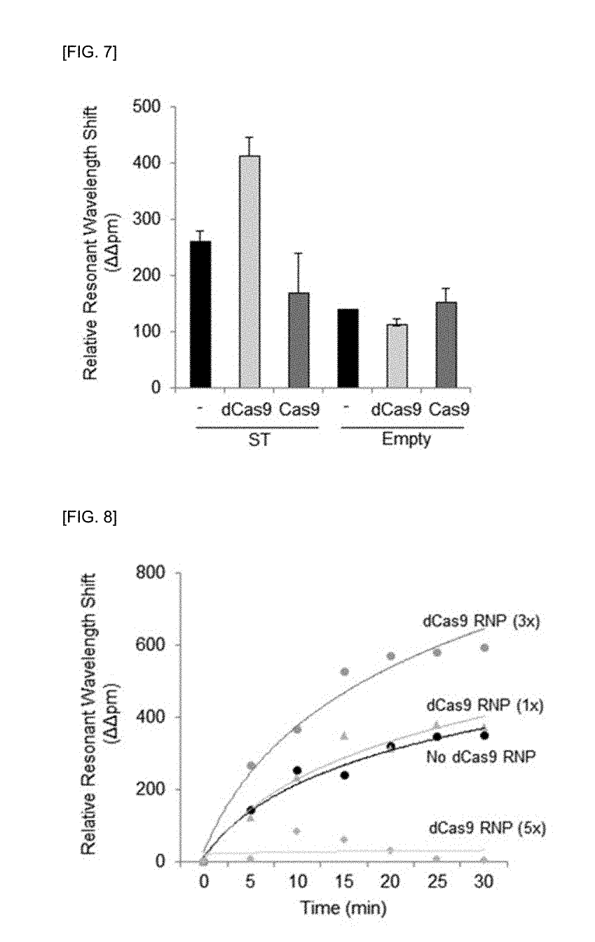

Also, in order to investigate whether dCas9 RNP improves detection sensitivity in SMR biosensors, DNA fragments was amplified from ST samples and signal enhancement was observed in dCas9 RNP-treated ST, which was compared with ST alone and ST treated with Cas9 RNP and as shown in FIG. 6, the detection sensitivity of ST treated with dCas9 RNP was improved. This sensitivity enhancement was due to the specific binding of dCas9 RNP to the target fragment on the SMR biosensor, the amplification efficiency is doubled than the conventional one and the difference between the non-target and the target was increased at least four times. In particular, the non-specific amplification of the non-target was prevented and the sensitivity and specificity were improved remarkably (FIG. 7).

In addition, as shown in FIG. 8, dCas9 RNP was treated by concentration to improve detection sensitivity of pathogenic nucleic acid, and 3×dCas9 RNP (300 ng of dCas9+225 ng of gRNA) showed the highest detection efficiency of pathogenic nucleic acid. [1×dCas9 RNP (100 ng dCas9+75 ng gRNA), 3×dCas9 RNP (300 ng dCas9+225 ng gRNA), 5×dCas9 RNP (500 ng dCas9+375 ng gRNA)]

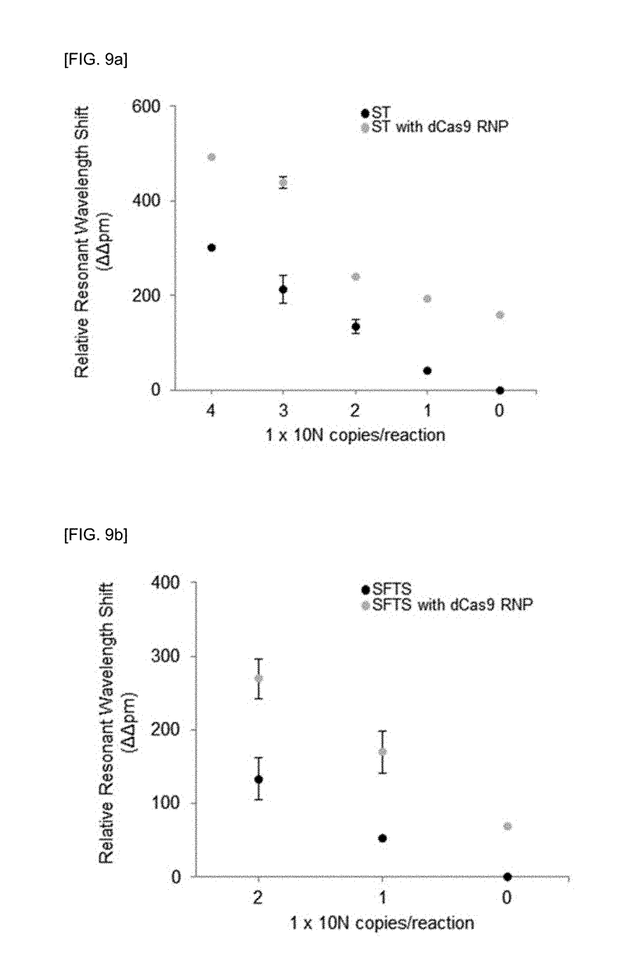

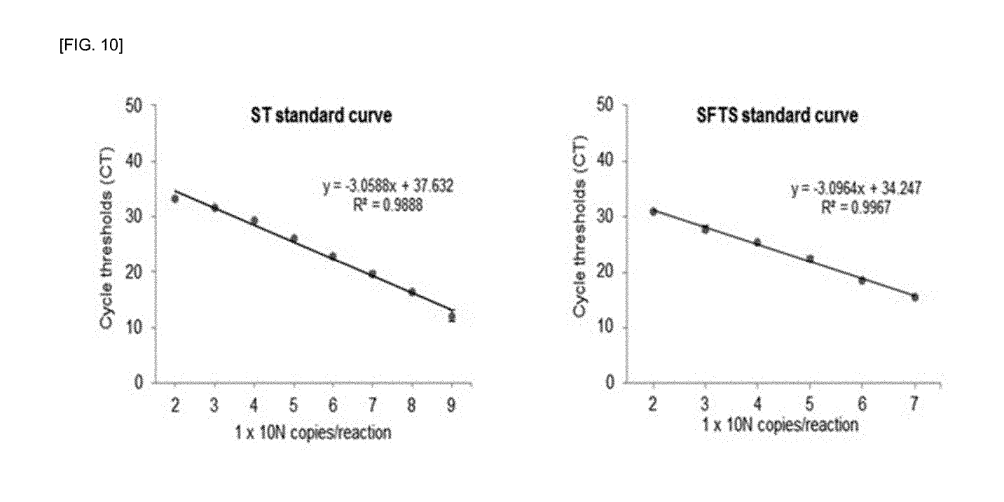

The detection limit of the CRISPR-mediated biosensor according to the present invention was confirmed to be detected as ST (0.54 aM) and SFTS (0.63 aM) within 30 minutes using dCas9 RNP as shown in FIG. 9A and FIG. 9B, respectively. This detection limit was confirmed to be superior to the detection limit of the SMR biosensor (˜10 copies) (FIG. 9a, FIG. 9b) and real time PCR (˜100 copies) (FIG. 10).

Therefore, using the CRISPR-mediated biosensor according to the present invention, pathogenic nucleic acids such as ST and SFTS could be detected more sensitively than SMR biosensors alone or real-time PCR methods.

### Example 2

### Clinical Sample Analysis Using CRISPR-Mediated Biosensor

The present inventors have investigated whether the CRISPR-mediated biosensor according to the present invention is useful for clinical application in a novel infectious disease requiring promptness, high sensitivity specificity. Because clinical symptoms of SFTS and ST substantially overlaps, molecular diagnostic tests to distinguish them early are important and are essential to provide ST-specific antimicrobial treatments and appropriate preventative measures for SFTS.

Thus, in the present invention, clinical samples were selected from a total six patients consisting of three patients with ST and three patients with SFTS.

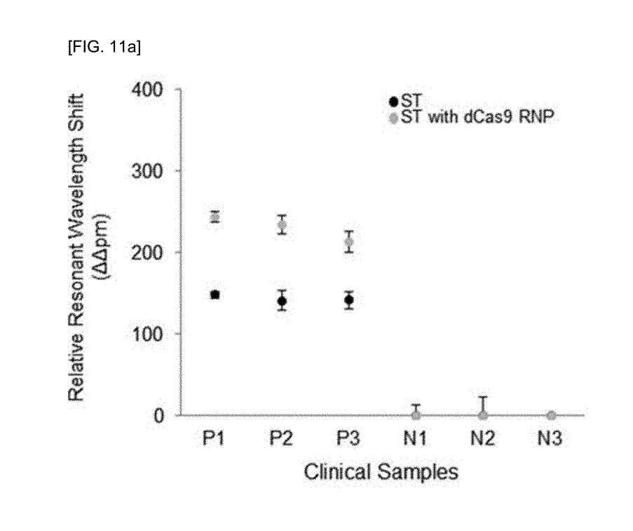

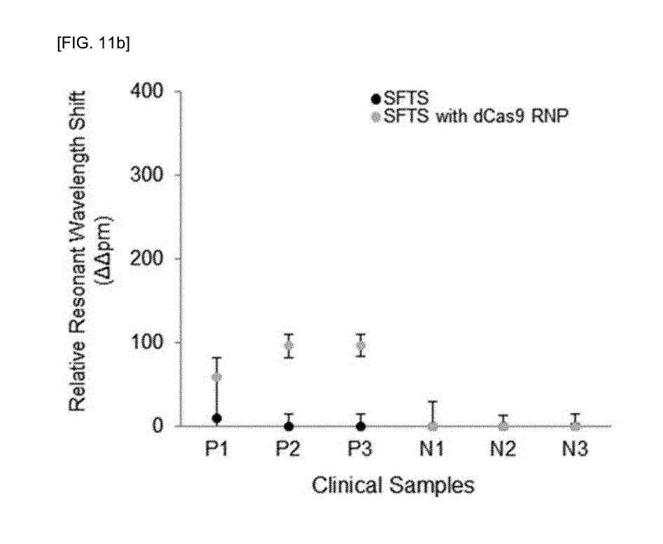

As a result of analyzing the diagnostic sample of ST with a CRISPR-mediated biosensor having an ST primer, an improved signal was observed only in the ST sample, not in the SFTS sample, as shown in FIG. 11A. In addition, as a result of analyzing these samples by a CRISPR-mediated biosensor having a SFTS primer to diagnose SFTS, an improved signal was observed only in the SFTS sample, as shown in FIG. 11B.

Therefore, the CRISPR-mediated biosensor according to the present invention can quickly and sensitively and specifically clearly distinguish ST and SFTS from clinical samples.

1. (canceled)

2. A method for improving sensitivity and specificity of nucleic acid detection, comprising:
providing a composition comprising dCas9 (dead Cas, nuclease-inactive Cas9) protein and gRNA (guide RNA) binding to a target nucleic acid sequence, as an active ingredient.

3. The method of claim 2, wherein the dCas9 protein is represented by an amino acid sequence of SEQ ID NO: 1.

4. The method of claim 2, wherein the target is a causative organism of an infectious disease selected from the group consisting of Orientia tsutsugamushi (OT), Bunyavirus, Mycobacterium tuberculosis, Mers virus and respiratory virus.

5. The method of claim 2, wherein the composition further comprises a nucleic acid polymerase, a primer capable of amplifying a target nucleic acid and a buffer solution.

6. The method of claim 2, wherein the nucleic acid is DNA or RNA.

7. A kit for improving sensitivity and specificity of nucleic acid detection comprising the composition of claim 2.

8. A method of improving sensitivity and specificity of nucleic acid detection, comprising:
amplifying a target nucleic acid by adding a dCas9 protein and a gRNA (guide RNA) binding to a target nucleic acid sequence to a sample containing the target nucleic acid; and
detecting an amplified target nucleic acid amplification product.

9. The method of improving sensitivity and specificity of nucleic acid detection of claim 8, wherein the dCas9 protein is represented by an amino acid sequence of SEQ ID NO:

1.

10. The method of improving sensitivity and specificity of nucleic acid detection of claim 8, wherein the target nucleic acid is amplified by using at least one selected from the group consisting of PCR, real-time PCR (RT-PCR), reverse transcriptase PCR, isothermal nucleic acid amplification and Silicon Microring Resonator (SMR).

11. The method of improving sensitivity and specificity of nucleic acid detection of claim 8, wherein the nucleic acid is DNA or RNA.
